(19) United States
(12) Patent Application Publication
(10) Pub. No.: US 2005/0148053 A1
(43) Pub. Date: Jul. 7, 2005

(54) Method for extraction of glycosaminoglycan from animal tissue

(75) Inventors: Isaac Ashie, Raleigh, NC (US); Bent Pill Pedersen, Bagsaverd (DK)

Correspondence Address: NOVOZYMES NORTH AMERICA, INC., 500 FIFTH AVENUE, SUITE 1600, NEW YORK, NY, 10110, US

(73) Assignees: Novozymes A/S, Bagsvaerd (DK); Novozymes North America, Inc., Franklinton, NC (US)

(21) Appl. No.: 10/970,169

(22) Filed: Oct. 21, 2004

Related U.S. Application Data
(60) Provisional application No. 60/513,034, filed on Oct. 21, 2003.

(51) Int. Cl. C08B 37/10; C12P 19/28; C08B 37/00

(52) U.S. Cl. 435/85; 536/53; 536/21

(57) ABSTRACT

The present invention relates to a method for extracting glycosaminoglycan from animal tissue by hydrolysing the tissue with endoprotease and exoprotease and separating the glycosaminoglycan.

### CROSS-REFERENCE TO RELATED APPLICATIONS

This application claims priority or the benefit under 35 U.S.C. 119 of U.S. provisional application No. 60/513,034 filed Oct. 21, 2003, the contents of which are fully incorporated herein by reference.

### TECHNICAL FIELD

The present invention relates to a method for extraction of glycosaminoglycan from animal tissue by hydrolysis of the animal tissue with protease and separation of the glycosaminoglycan from the hydrolysed animal tissue.

### BACKGROUND OF THE INVENTION

Cartilage and connective tissue contain significant amounts of proteoglycans which are composed of glycosaminoglycan attached to a linear core protein. The glycosaminoglycans have been shown to elicit chondroprotective (e.g., inhibit cartilage degradation, stimulate collagen and proteoglycan synthesis, etc), anti-atherosclerotic, moisturizing, and other medicinal effects. These have provided the incentive for their commercial extraction from various sources. Some of the sources include trachea, cartilage, rooster combs, and intestinal mucosa, from pigs, cattle, and poultry. The major glycosaminoglycans found in cartilage and other sources are chondroitin sulphate, hyaluronate, keratan sulphate, dermatan sulphate, heparin, and heparan sulphate. The extraction process involves breakdown of the surrounding tissues and core proteins by alkaline hydrolysis to release the glycosaminoglycan which are then subjected to further downstream processing for recovery. However, alkaline hydrolysis requires significant amounts of water for downstream processing and therefore raises environmental concerns as well as processing cost. The use of proteolytic enzymes alleviates some of the drawbacks of alkaline hydrolysis.

JP 05125103 discloses the use of protease for hydrolysis of rooster comb for extraction of hyaluronate.

### SUMMARY OF THE INVENTION

The inventors found that hydrolysing animal tissue with endoprotease and exoprotease was more effective when extracting glycosaminoglycan from animal tissue than hydrolysis with endoprotease alone.

The invention thus relates to a method for extracting glycosaminoglycan from animal tissue comprising
a) hydrolysing the tissue with endoprotease and exoprotease; and
b) separating glycosaminoglycan from the hydrolysed animal tissue.

### DETAILED DESCRIPTION OF THE INVENTION

Glycosaminoglycan

Glycosaminoglycans are long unbranched polysaccharides containing a repeating disaccharide unit. The disaccharide units contain either of two modified sugars, N-acetylgalactosamine (GalNAc) or N-acetylglucosamine (GlcNAc), and a uronic acid such as glucuronate or iduronate. Glycosaminoglycans are highly negatively charged molecules with extended conformation located primarily on the surface of cells or in the extracellular matrix. Low compressibility of glycosaminoglycans makes these molecules ideal as lubricating fluid in the joints. At the same time, their rigidity provides structural integrity to cells and passageways between cells, allowing for cell migration. The major glycosaminoglycans found in animal tissue are hyaluronate, dermatan sulfate, chondroitin sulfate, heparin, heparan sulfate, and keratan sulfate. Although each of these glycosaminoglycans has a predominant disaccharide component, heterogeneity does exist in the sugars present in the make-up of any given class of glycosaminoglycan. Hyaluronate is unique among the glycosaminoglycans in that it does not contain any sulphate.

In a preferred embodiment, the invention relates to a method for extraction of hyaluronate, dermatan sulfate, chondroitin sulfate, heparin, heparan sulfate, and/or keratan sulphate.

Proteases

Two sets of subclasses of proteases are recognised, those of the exoproteases and those of the endoproteases. The exoproteases act near the ends of polypeptide chains, and those acting at a free N-terminus liberate a single amino-acid residue, a dipeptide and/or a tripeptide. The exoproteases acting at a free C-terminus liberate a single residue and/or a dipeptide. In the present context an exoprotease is an enzyme that exhibits exoprotease activity. Endoproteases act internally in peptide chains. In the present context an endoprotease is an enzyme that exhibits endoprotease activity. For classification of proteolytic enzymes see e.g. Handbook of Proteolytic Enzymes, Barret et al., eds., Academic Press, London 1998.

Endoprotease

An endoprotease according to the invention may be any endoprotease, e.g. a serine endoprotease, a cysteine endoprotease, an aspartic endoprotease, or a metalloprotease; or any combination of endoproteases. The endoprotease may be of any origin, e.g. of microbiological origin, such as of bacterial or fungal origin. In a preferred embodiment the endoprotease is derived from Bacillus.

In one embodiment of the invention the endoprotease comprises a serine endoprotease. In a preferred embodiment the endoprotease is a serine endoprotease. A serine endoprotease is an endoprotease wherein the catalytic mechanism depends upon the hydroxyl group of a serine residue. Examples of serine endoproteases are e.g. trypsin, chymotrypsin, and subtilisin. A serine endoprotease according to the invention is e.g. Subtilisin 309 described in WO 89/06279.

In another embodiment of the invention, the endoprotease comprises a metalloprotease. In a preferred embodiment the endoprotease is a metalloprotease. A metalloprotease according to the invention is a protease that requires zinc or metal ions for catalysis. In a preferred embodiment the metalloprotease belongs to peptidase family M4, also known as thermolysin-like proteases. In a further preferred embodiment the metalloprotease is derived from Bacillus, e.g. from B. amyloliquefaciens. An example of a metalloprotease according to the invention is Bacillolysin, e.g. Bacillolysin from Bacillus amyloliquefaciens.

In a preferred embodiment of the invention the endoprotease comprises a serine endoprotease and a metalloprotease. In a more preferred embodiment the endoprotease consists of a serine endoprotease and a metalloprotease.

In one embodiment of the invention an endoprotease, e.g. a serine endoprotease, is dosed at e.g. 10-400, such as 40-200, or 80-130 KNPU/kg raw material (KNPU: Kilo Novo Protease unit. Assay description is available from Novozymes AIS, Bagsvaerd, Denmark).

In a further embodiment of the invention an endoprotease, e.g. a metallo protease, is dosed at e.g. 0.1-6.0, such as 0.4-3.2, or 0.6-1.6 AU/kg raw material (AU: Anson unit. A measure of the rate of degradation of denatured haemoglobin by the enzyme under incubation at pH 7.5, 25° C. for 10 min. See e.g. Analytical Biochemistry vol. 100, pp. 201-220, 1979).

Exoprotease

An exoprotease according to the invention may be any exoprotease (e.g. an aminopeptidase, dipeptidyl-peptidase, tripeptidyl-peptidases, carboxypeptidase, or peptidyl-dipeptidase) or combination of exoproteases. The exoprotease may be of any origin, e.g. of microbial origin, such as e.g. bacterial or fungal origin. In a preferred embodiment the exoprotease is derived from Aspergillus, e.g. from A. oryzae. An example of an exoprotease according to the invention is the serine carboxypeptidase described in WO 9814599.

The exoprotease dose may e.g. be 100-2000, such as 200-1000, or 300-800 LAPU/kg raw material (LAPU: Leucine aminopeptidase unit. 1 LAPU is the amount of enzyme which hydrolyzes 1 mmol L leucine-p-nitroanilide per minute in 0.1 M Tris buffer at pH 8.0, 40° C. See e.g. Bergmeier, Methods Enzyme Anal. 5,11-15 1984).

The enzymes used in the invention may be purified. The term “purified” as used herein covers enzyme protein where components from the organism from which it is derived have been removed. The term “purified” may also cover enzyme protein where components from the native organism from which it is obtained have been removed.

Animal tissue

Animal tissue according to the invention may be any material derived from an animal such as e.g. muscle, bone, joint, skin, tendon, internal organ, intestine, blood, blood vessel, connective tissue, trachea, cartilage, rooster comb, intervertebral disc, heart valve, cornea, fish fin, and intestinal mucosa. Animal tissue may be derived from any kind of animal such as e.g. pig, cattle, poultry, fish, and shellfish. The animal tissue may be pre-treated in any suitable way before hydrolysis, e.g. by rinsing, trimming, grinding, suspension in water, and/or heat treatment.

Hydrolysis

Hydrolysis may be conducted under any conditions suitable to achieve desired enzymatic activity. Usually the animal tissue to be hydrolysed will be mixed with water. pH of the suspension may be adjusted and will usually be in the range 6-10, depending e.g. on the pH optimum of the proteolytic enzymes. Hydrolysis temperature will usually be between 30 and 80° C. Usually a high degree of protein breakdown is desired in order to achieve a high yield of glycosaminoglycan. In a preferred embodiment of the invention the degree of hydrolysis of the protein after hydrolysis is above 25%, such as above 30%, preferably above 32%, and most preferably above 34%. Degree of hydrolysis is defined as the percent of peptide bonds that has been hydrolysed. The hydrolysis reaction may be terminated by inactivation of the enzyme when a preset time or a desired extent of hydrolysis has been achieved. Inactivation of the enzymes may be conducted by any method known in the art. In a preferred embodiment the enzymes are inactivated by heating at a temperature and time sufficient to inactivate the enzymes, such as e.g. at 60-100° C. for 1-120 minutes.

Separation of Glycosaminoglycan

Glycosaminoglycan can be separated from the crude extract obtained from hydrolysis using methods known in the art, e.g. by washing with water, ion-exchange and lyophilisation. The crude extract may be de-fatted with organic solvents followed by further purification before separation of glycosaminoglycan.

The present invention provides a method with increased efficiency for extraction of glycosaminoglycan from animal tissue, e.g. the yield and/or purity of glycosaminoglycan may be increased compared to the methods of the prior art.

### EXAMPLES

### Example 1

### Hydrolysis of Cattle Trachea

400 g of cattle trachea was hydrolysed under the following conditions:

| | |
|---|---|
| Substrate concentration: | 400 g trachea + 400 g water |
| Temperature: | 55° C. |
| pH: | natural |
| Hydrolysis time: | 16 hours |
| Inactivation of enzymes: | heating at 85° C. for 15 minutes. |

Enzymes used for hydrolysis:
Serine endoprotease: Subtilisin 309 (described in WO 89/06279).
Metalloendoprotease: Bacillolysin from Bacillus amyloliquefaciens.
Exoprotease: A fermentation broth supernatant of the Aspergillus oryzae strain ATCC20386 with exoprotease activity mainly originating from a serine carboxypeptidase (described in WO 9814599), activity: 500 LAPU/g
Commercial endoprotease preparation derived from Bacillus (Biocatalysts, UK) 235 CPU/g

The following enzyme combinations were used:

| | | | Exoprotease |
|---|---|---|---|
| | Serine endoprotease | Metalloprotease | (LAPU/kg |
| Combination no. | (KNPU/kg trachea) | (AU/kg trachea) | trachea) |
| 1 | 96 | 1.6 | 0 |
| 2 | 96 | 0.8 | 500 |
| Commercial | | | |
| endoprotease | | | |
| (1645 CPU/kg | | | |
| trachea) | | | |

After hydolysis the degree of hydrolysis in the supernatant (DH) was determined by the improved method for determining food protein degree of hydrolysis (Nielsen, P. M., Petersen, D., and Dambmann, C. 2001. Improved method for determining food protein degree of hydrolysis. J. Food Sci. 66(5): 642-646.)

The concentration of protein in the supernatant was measured by Leco protein analyzer using a conversion factor of 6.25

Osmolality was measured by Osmometer.

Results are shown in table 1.

Visual inspection of the hydrolysis product showed more extensive breakdown of connective tissue and therefore less residual tissue attached to the stirrer blades when endoprotease and exoprotease (combination 2) was used compared to the commercial endoprotease preaparation.

TABLE 1
Degree of hydrolysis (DH) and protein concentration in supernatants.

| Combination | pH at | pH at | Osmolality | | Protein |
|---|---|---|---|---|---|
| no. | start | end | (mOsm/kg) | DH (%) | (% of supernatant) |
| 1 | 6.64 | 6.20 | 438 | 30.45 | 11 |
| 2 | 6.54 | 6.37 | 507 | 37.08 | 10 |
| Commercial | 6.79 | 6.23 | 413 | 29.90 | 10 |
| endoprotease | | | | | |

1. A method for extracting glycosaminoglycan from animal tissue comprising
a) hydrolysing the tissue with endoprotease and exoprotease; and
b) separating glycosaminoglycan from the hydrolysed animal tissue.

2. The method of claim 1 wherein the endoprotease comprises a serine endoprotease.

3. The method of claim 1 wherein the endoprotease comprises a metalloprotease.

4. The method of claim 1 wherein the endoprotease comprises a bacterial endoprotease.

5. The method of claim 4 wherein the bacterial endoprotease is derived from Bacillus.

6. The method of claim 1 wherein the exoprotease comprises a fungal exoprotease.

7. The method of claim 6 wherein the fungal exoprotease is derived from Aspergillus.

8. The method of claim 1 wherein the glycosaminoglycan is one or more of chondroitin sulphate, hyaluronate, keratin sulphate, dermatan sulphate, heparin, and heparan sulphate.
